(19) United States
(12) Patent Application Publication
(10) Pub. No.: US 2004/0009134 A1
(43) Pub. Date: Jan. 15, 2004

(54) Hair detangler and light conditioner

(76) Inventor: Bernard Bendiner, Pinehurst, NC (US)

Correspondence Address: BRINKS HOFER GILSON & LIONE, P.O. BOX 10395, CHICAGO, IL, 60611, US

(21) Appl. No.: 10/193,726

(22) Filed: Jul. 11, 2002

(51) Int. Cl. A61K 7/06

(52) U.S. Cl. 424/70.1

(57) ABSTRACT

A method for producing an all natural and organic hair detangler and light conditioner that is odorless and crystal clear. This detangler and conditioner can be used for human hair or the fur of domestic animals. The product has colloidal sized particles of cellulose bonded by wax that are suspended in the solution. The process for producing this detangler and conditioner includes the step of placing special wax paper in a filter bag, similar to but larger than a tea bag, immersing the filter bag in hot water causing infinitesimal sized particles of cellulose bonded by wax to pass through the filter and to become suspended in the water. The special wax paper is produced by the dry wax process in which pressure is applied to impregnate the wax into the tissue paper. In the preferred embodiment of this invention the wax on the wax paper is bees wax. The filtrate from this initial filter is passed through a second finer filter which provides a finished aqueous product that is crystal-clear, odorless and contains colloidal particles of cellulose bonded by wax.

### BACKGROUND OF THE INVENTION

The present invention relates to cosmetology, body-hygiene and, in general, a product that can be used for the hair and skin of humans or the fur and skin of domestic animals.

There are available hair detanglers and light conditioners that have some natural constituents but none are known that have a total 100% all-organic and all-natural ingredients. Products made from all natural product that provide the desired results and perform in an ideal fashion are desired by and provide major benefits to those who prefer not to use drug-like products and those who are allergic to artificial products.

### BRIEF SUMMARY OF THE INVENTION

The product of this invention is odorless and crystal clear and has invisible particles of cellulose bonded by wax suspended in the water.

The product according to this invention has three functions. First it serves to protect, maintain, and in some situations even restores, the biological and physiological function of hair and fur. A second function consists in creating a suitable stable pharmaceutical and serves to prevent snarls and tangles. A third function consists of treating hair or fur as well as the underlying skin by providing it with one or more topical benefits, in particular a therapeutic and or cosmetic benefits.

The process or method for making this product consists of utilizing a certain type of wax paper containing a certain type of wax; and a unique method of placing this wax in solution. The cellulose from the paper as well as the wax are utilized in the final product of this invention. The method involves placing the special wax paper in a filter bag, similar to but larger than a tea bag, immersing the filter bag in hot water causing infinitesimal sized particles of cellulose bonded by wax to pass through the filter and to become suspended in the water. This phenomenon causes the water to retain a minimal amount of wax which facilitates and enables the detangling effect of the hair or fur. Furthermore, the invisible size colloidal wax cellulose particle adds to the detangling and hair conditioner function as a result of the cellulose acting as a micro-colloidal brush and the wax functioning as a lubricant between the strands of hair and fur. This filtrate is then passed through a second finer filter to remove any cellulose fibers or other foreign matter that might have been contained in the tissue paper and was of a size that could pass through the initial filter but is larger than the colloidal particles of cellulose bonded by wax. The finished aqueous product is crystal-clear, odorless and contains colloidal particles of cellulose bonded by the wax. This process is important to applicant's invention since if the dry wax tissue is added directly to the water in the blending tank it forms an opaque finished product that is sticky, and does not function as a hair detangler and light conditioner.

One of the highlights of this invention is that no preservative is necessary to add to the liquid product even though it is an all-natural and all-organic liquid product. Conventional wisdom leads one to believe that a preservative would have to be added to this to prevent molding. However, an unexpected result of this product is that it maintains its all-natural and organic quality as a result of the wax functioning as a preservative and preventing the formation of mold. It is a natural substance in which the mold microorganisms are prevented from becoming viable.

### DETAILED DESCRIPTION OF THE INVENTION

The process for making this product consists of utilizing a certain type of wax paper containing a certain type of wax; which is processed by a unique method to place the wax in solution.

The wax paper used in this process is similar to a type normally used in bakeries to place cookies and other bakery items on, prior to selling and for packaging the product for the customer. In the manufacturer of this wax paper for bakeries a wet strength resin is normally used to provide some moisture resistance to the paper. That wet strength resin is not applied to the paper that is used for this process. Also, the bakery paper uses paraffin wax which can be used in this process, however in applicant's preferred embodiment bees wax is used.

This type of wax paper is commonly called “tissue,” and in the paper industry is referred to as 10¾ Pound Basis Weight wax paper. This commercial designation means that a ream of this wax paper has 500 sheets that are 24 by 36 inches and the paper alone weighs approximately 10¾ pounds. The 500 sheets of tissue in this ream of paper have been sprayed with about 3½ pounds of wax and thus the ream actually weighs about 14¼ pounds. This type of wax paper is made by a process called the dry wax application, in which wax is sprayed on the surface of the tissue paper under pressure such that the wax impregnates into the surface of the tissue paper. Although 10¾ pound basic weight tissue paper is commercially available and is used for applicant's process, the weight of the paper is not important to applicant's invention and tissue paper of other weights could be used. For example, it could be made at either a higher or lower basis weight. The commercially available 10¾ basic weight tissue paper that is used in bakery and deli shops has paraffin wax applied to the tissue paper.

Although paraffin wax is made from products that are derived from the earth and thus could be considered to be a natural and organic product, paraffin wax is not ingestible. For this reason in applicant's preferred embodiment he has the tissue paper made with bees wax rather than paraffin wax. Bees wax is an all-organic and all-natural product that is ingestible, as is honey that is also produced by bees. When applicant's product is sprayed on the hair the mist could be ingested through the mouth or nose. For this reason applicant prefers to use bees wax in his product because it renders his product safer than if the product is made from paraffin type wax.

Common wax paper, for example wax paper that can be purchased in a grocery store is not used in applicant's process. The wax in common wax paper is applied to the sheet of paper by the wet wax process and as a result the wax is not impregnated into the fiber but rather is applied to the sheet of paper as a layer on top of the sheet. In the “dry wax process” used to produce the paper used for cookies and cakes and in applicant's process, the wax is applied to the sheet of tissue under pressure and the wax impregnates the surface of the tissue. The use of wax paper that has had the wax applied by the dry wax process is important to applicant's invention because during applicant's process for forming his solution it is important that wax has impregnated into the surface of the tissue paper.

Applicant's method of producing the solution of this invention involves placing the special wax paper in a filter bag, similar to but larger than a tea bag, immersing the filter bag in hot water causing infinitesimal sized particles of cellulose bonded by wax to pass through the filter and to become suspended in the water. The process is performed in a conventional blending tank having a low rpm agitator to produce a slight movement of the water causing the heated water to pass through the filter bag containing the wax paper and carrying with it into the water outside of the filter bag the colloidal sized wax bound cellulose particles.

Although tap water or deionized water can be used for this process, applicant uses reverse osmosis water for producing the preferred embodiment of his product, because the finished product is going onto the hair of humans or pets and the purer the water, the more effective is the product.

The use of wax paper that has had the wax applied by the dry wax process is important to applicant's invention because during applicant's step of immersing the filter bag in hot water the wax that permeated the surface of the tissue has bonded with colloidal sized particles of cellulose. These colloidal sized particles of cellulose that are bonded by a wax film become free in the hot water of the blending tank. The filter permits these colloidal sized particle to pass through and prevents the substantially larger particles from transcending the filter. The colloidal sized particles of cellulose that are bonded with a wax that do pass through the filter are suspended in the aqueous solution and do not settle out. Because of the minute size of the particles of cellulose that are bonded with wax they are invisible to the naked eye and the solution appears clear. Although the particles of cellulose that are bonded with wax are very small they are the major reason for the effectiveness of the invention to detangle and lightly condition the hair of adults, children and pets. In the commercial process this filtrate is passed through a second finer filter to remove any particles that are larger than the particles of cellulose that are bonded with wax to thus provide a clear final product.

The initial filter used by applicant is similar to a tea bag but much larger. Applicant has used a filter bag having a draw-string at the top. The wax paper is placed into the filter bag through the open top, securely close the bag through the draw-string and then immerse the filled filter bag into the water. The temperature of the water must be at 140 F. or higher because the wax melts at this temperature. The phenomenon of this process and product is that it does not recrystallize when transferred from a hot to a cold system. In other words, the wax that is bonded to the colloidal sized cellulose particles does not recrystallize. The openings in the filter are of a size that will permit the colloidal sized wax bound cellulose particles permeates through the filter, as tea does through a tea bag, and the larger particles are retained in the filter bag. Applicant has found that a filter bag having openings in the range of 75 to 200 microns performs well for this function however applicant uses a 100 micron filter in his preferred embodiment. This filtrate is then passed through a finer filter (openings 1-8 microns) which in the preferred embodiment has openings of 5 microns.

The finished product is effective as a light conditioner and detangler for hair and fur at concentrations of as low as 1% solid matter and 99% water. Thus for a low concentration such as this in a gallon of water which weighs 3,784 grams there can be as little as 1% or 37 grams of wax. Thus 37 grams of wax from the dry wax tissue that is placed into the filter bag must be bonded to colloidal sized particles of cellulose for each gallon of product to be produced. These 37 grams of wax will not be in a crystallized form when the temperature of the final product falls below the melting temperature of wax and substance changes from a hot to a cold system. This is a very significant phenomenon of applicant's process for producing the final product. As a result of this phenomenon the finished product is crystal-clear and has no odor.

However, applicant has found that the product is even more effective at higher concentrations and detangles and manages better if the ratio of wax is increased and applicant's preferred embodiment uses about 6% by weight of dry wax tissue in the initial filter and 94% water by weight.

For a final product containing 1% wax you need about 37 grams of wax per gallon of water since one gallon of water weighs 3,784 grams. However, for a final product having 1% wax more than 37 grams of dry wax tissue must be placed in the filter for each gallon of water because about ⅔ of the mass of the dry wax tissue mass will remain within the filter and only about ⅓ will pass through the filter as a wax film on the colloidal sized particles of cellulose. Applicant's preferred embodiment has about 2% wax and thus it requires about 76 grams of solid per gallon of water. The solid includes both wax and cellulose, however the weight of the cellulose is infinitesimal.

### EXAMPLE

If it is desires to create 10 gallons of final product the following is placed in the blending tank:

10 gallons of water (37,840 grams) and

A 75-150 micron filter containing about 2,270 grams of dry wax tissue.

About ⅓ of the dry wax tissue will pass through the filter as colloidal sized particles of cellulose with a wax film and be suspended in the water outside of the filter. This filtrate will then be subjected to a second finer filter (opening 1-8 microns). Thus, the final product will contain about 2% wax (2,270/3÷37840). Therefore, this final product from the starting ingredients that include about 6% by weight of dry wax tissue has about 98% water and 2% solids. The lost ingredients remain in the filter bag and are discarded.

This hair detangler and light conditioner can be sprayed directly to the hair or fur or can be incorporated into a shampoo and applied while washing the hair or fur.

The foregoing specification describes only preferred embodiments of the invention as disclosed. Other embodiments besides the ones described above may be articulated as well. The terms and expressions therefore serve only to describe the invention by example only and not to limit the invention. It is expected that others perceive differences which while differing from the foregoing, do not depart from the spirit and scope of the invention herein described and claimed.

1. A process of producing a hair and fur detangler and conditioner comprising the steps of:
providing a filter bag having openings in the range of 75-200 microns in size;
placing wax paper in the filter bag;
providing a blending tank containing water at a temperature sufficient to melt wax;
submerging the filter bag containing wax paper in the blending tank containing water at a temperature sufficient to melt wax; and
blending the water in the blending tank causing it to pass through the surfaces of the filter bag and permeate throughout the blending tank causing colloidal sized cellulose particles bonded by wax to become suspended in the water outside of the filter bag.

2. A process of producing a hair and fur detangler and conditioner as set forth in claim 1 wherein:
the wax paper that is placed in the filter bag is of the type produced by the dry wax process dry wax tissue type.

3. A process of producing a hair and fur detangler and conditioner as set forth in claim 1 wherein:
the water provided in the blending tank is at least 140° F.

4. A process of producing a hair and fur detangler and conditioner as set forth in claim 1 wherein:
the wax paper that is placed in the filter bag is 10¾ pound basis weigh tissue paper.

5. A process of producing a hair and fur detangler and conditioner as set forth in claim 1 wherein:
the wax on the wax paper that is placed in the filter bag is bees wax.

6. A process of producing a hair and fur detangler and conditioner as set forth in claim 1 wherein the following additional step is performed:
passing the filtrate through a second finer filter.

7. A process of producing a hair and fur detangler and conditioner as set forth in claim 6 wherein:
the second finer filter has openings in the range of 1-8 microns.

8. A process of producing a hair and fur detangler and conditioner as set forth in claim 1 wherein:
the colloidal sized cellulose particles bonded by wax contained in the final product constitute about 1% by weight of the final product.

9. A process of producing a hair and fur detangler and conditioner as set forth in claim 1 wherein:
the colloidal sized cellulose particles bonded by wax contained in the final product constitute about 2% by weight of the final product.

10. A process of producing a hair and fur detangler and conditioner as set forth in claim 3 wherein:
the wax paper that is placed in the filter bag is of the type produced by the dry wax process dry wax tissue type.

11. A process of producing a hair and fur detangler and conditioner as set forth in claim 4 wherein:
the wax paper that is placed in the filter bag is of the type produced by the dry wax process dry wax tissue type.

12. A process of producing a hair and fur detangler and conditioner as set forth in claim 5 wherein:
the wax paper that is placed in the filter bag is of the type produced by the dry wax process dry wax tissue type.

13. A process of producing a hair and fur detangler and conditioner as set forth in claim 6 wherein:
the wax paper that is placed in the filter bag is of the type produced by the dry wax process dry wax tissue type.

14. A process of producing a hair and fur detangler and conditioner as set forth in claim 9 wherein:
the wax paper that is placed in the filter bag is of the type produced by the dry wax process dry wax tissue type.

15. A process of producing a hair and fur detangler and conditioner as set forth in claim 2 wherein:
the colloidal sized cellulose particles bonded by wax contained in the final product constitute about 2% by weight of the final product.

16. A process of producing a hair and fur detangler and conditioner as set forth in claim 3 wherein:
the colloidal sized cellulose particles bonded by wax contained in the final product constitute about 2% by weight of the final product.

17. A process of producing a hair and fur detangler and conditioner as set forth in claim 4 wherein:
the colloidal sized cellulose particles bonded by wax contained in the final product constitute about 2% by weight of the final product.

18. A process of producing a hair and fur detangler and conditioner as set forth in claim 5 wherein:
the colloidal sized cellulose particles bonded by wax contained in the final product constitute about 2% by weight of the final product.

19. A process of producing a hair and fur detangler and conditioner as set forth in claim 6 wherein:
the colloidal sized cellulose particles bonded by wax contained in the final product constitute about 2% by weight of the final product.

20. A process of producing a hair and fur detangler and conditioner as set forth in claim 19 wherein:
the second finer filter has openings in the range of 1-8 microns.
